(19) United States
(12) Patent Application Publication
(10) Pub. No.: US 2025/0263648 A1
(43) Pub. Date: Aug. 21, 2025

(54) DYNAMIC NUTRIENT CONTROL PROCESSES

(71) Applicant: Janssen Biotech, Inc., Horsham, PA (US)

(72) Inventors: James Vincent PRICE, Ardmore, PA (US); Stephanie BERGES-VORSANGER, Dresher, PA (US); Amalie LEVY, Ardmore, PA (US); Steven SAVAGE, Royersford, PA (US); Abbey WEITH, Springfield, PA (US)

(21) Appl. No.: 18/555,205

(22) PCT Filed: Apr. 12, 2022
(86) PCT No.: PCT/US2022/024404

§ 371 Date: Oct. 12, 2023

Related U.S. Application Data
(60) Provisional application No. 63/174,143, filed on Apr. 13, 2021.

(51) Int. Cl. C12M 1/34; C12M 1/00

(52) U.S. Cl. CPC C12M 41/32; C12M 29/00; C12M 41/36

(57) ABSTRACT

Materials and methods to control a nutrient feed in a cell culture process is provided. A sample is received from a bioreactor comprising a cell culture. A residual amount of nutrient is determined from the received sample. A cellular growth rate between a current culture day and a previous day is calculated. An integrated viable cell density for a next day is predicted. A nutrient target for the next day is calculated. The nutrient is fed to the bioreactor according to the calculated nutrient target.

### CROSS REFERENCE TO RELATED APPLICATION

This application is a Patent Cooperation Treaty (PCT) application of, and claims priority under Article 8 of the PCT to, U.S. Provisional Application No. 63/174,143 filed on Apr. 13, 2021, the entirety of which is incorporated herein as if fully set forth below.

### BACKGROUND

Cell culture productivity relies on optimization of culture medium management enabling high cell densities. Nutrient feeding is an important parameter for process optimization. High cell density processes can require substantial amounts of nutrients and the daily requirements vary with cell types or density.

Glucose feeding is an important factor for bioreactor process optimization. High cell densities and productivity often implicate a need to feed substantial amounts of glucose (usually exceeding 10 g/L per day at peak cell density). Existing glucose feeding algorithms rely on historical databases which consume computing power and resources.

Daily glucose requirements for cell lines can vary greatly between cell lines. Changes in process parameters such as, production bioreactor target seeding density and basal media glucose concentration, can affect the daily glucose requirements.

A need exists for a cell culture solution that automates the nutrient feeding process, such as the glucose feeding process, without relying on historical database. There is also a need for a predictive cell-based glucose algorithm that takes into consideration of process parameter changes.

### SUMMARY

Improved materials and methods for the prediction of daily nutrient target requirements for a given cell line is needed and is addressed by the present invention. One aspect of the disclosed technology relates to a method of controlling a nutrient feed in a cell culture process. A sample may be received from a bioreactor comprising a cell culture. A residual amount of nutrient may be measured from the received sample. A variable cell density on a current day may be determined based on the measured residual amount of nutrient. A cellular growth rate between the current culture day and a previous day may be calculated based at least on the determined viable cell density on the current day. A viable cell density for a next day may be predicted based at least on the calculated cellular growth rate. An integrated viable cell density for the next day may be predicted based at least on the predicted viable cell density for the next day. A nutrient target for the next day may be calculated based at least on the predicted integrated viable cell density for the next day. Nutrient may be fed to the bioreactor according to the calculated nutrient target for the next day.

In one embodiment, the cellular growth rate between the current culture day and a previous day may be calculated based on the determined viable cell density on the current day, and a viable cell density measured on a previous day.

In one embodiment, the viable cell density for the next day may be predicted based on the calculated cellular growth rate and the determined viable cell density on the current day.

In one embodiment, the integrated viable cell density for the next day may be predicted based on the predicted viable cell density for the next day, the determined viable cell density on the current day, and an integrated viable cell density for the current day.

In one embodiment, a daily specific nutrient consumption rate over the previous day may be calculated based at least on the calculated cellular growth rate. An amount of nutrient to be consumed between the current day and the next day may be predicted based on the daily specific nutrient consumption rate. The nutrient target may be calculated based at least on the predicted amount of nutrient to be consumed.

In one embodiment, the daily specific nutrient consumption rate over the previous day may be calculated based on the calculated cellular growth rate, an integrated viable cell density for the current day, and an integrated viable cell density for the previous day.

In one embodiment, the amount of nutrient to be consumed between the current day and the next day may be predicted based on the daily specific nutrient consumption rate, the predict integrated viable cell density for the next day, and an integrated viable cell density for the current day.

In one embodiment, the nutrient target may be calculated based on the predicted nutrient to be consumed and an empirically determined nutrient to maintain value.

In one embodiment, one or more of the following: the viable cell density measured on the previous day, the integrated viable cell density for the current day, the integrated viable cell density for the previous day, and the empirically determined nutrient to maintain value, may be retrieved from a non-transitory computer readable medium.

In one embodiment, the nutrient may be selected from glucose, glutamate, galactose, lactate, and glutamine.

In one embodiment, the nutrient may include one or more monosaccharides.

In one embodiment, the residual nutrient measurement may include assaying a nutrient concentration in the bioreactor.

In one embodiment, the residual nutrient measurement may include performing one or more of offline nutrient measurement and inline nutrient measurement.

In one embodiment, the residual nutrient measurement may be performed by one or more of the following: a NovaFlex device and a Raman Probe.

In one embodiment, the bioreactor may be one or more of the following: a Chinese hamster ovary (CHO) cell bioreactor, and a 5 L bioreactor. Other mammalian cell types that may be used in manufacturing biologics besides CHO, including recombinant cells and the like. Non-limiting examples of such mammalian cell types include HEK, 293 and PerC6. This process may be also used for other non-mammalian cell types, such as, for example, yeast and bacteria.

In one embodiment, cells in the bioreactor may be mammalian cells.

In one embodiment, the cells may be CHO cells.

Another aspect of the disclosed technology relates to a method of controlling a glucose feed in a cell culture process. A sample may be received from a bioreactor comprising a cell culture. A residual amount of glucose may be measured from the received sample. A variable cell density on a current day may be determined based on the measured residual amount of glucose. A cellular growth rate between the current culture day and a previous day may be calculated based at least on the determined viable cell density on the current day. A viable cell density for a next day may be predicted based at least on the calculated cellular growth rate. An integrated viable cell density for the next day may be predicted based at least on the predicted viable cell density for the next day. A glucose target for the next day may be calculated based at least on the predicted integrated viable cell density for the next day. Glucose may be fed to the bioreactor according to the calculated glucose target for the next day.

Further features of the present disclosure, and the advantages offered thereby, are explained in greater detail hereinafter with reference to specific embodiments illustrated in the accompanying drawings, wherein like elements are indicated by like reference designators.

### BRIEF DESCRIPTION OF THE DRAWINGS

Reference will now be made to the accompanying drawings, which are not necessarily drawn to scale, and which are incorporated into and constitute a portion of this disclosure, illustrate various implementations and aspects of the disclosed technology and, together with the description, explain the principles of the disclosed technology. In the drawings:

FIG. 1 is a schematic diagram of an example environment that may be used to implement one or more embodiments of the present disclosure.

FIG. 2 is a schematic diagram of an example environment that may be used to implement one or more embodiments of the present disclosure.

FIG. 3 is a flow chart of glucose algorithm according to one aspect of the disclosed technology.

FIG. 4 is an example of an automated process for feeding glucose according to one aspect of the disclosed technology.

FIG. 5 is a block diagram of a nutrient feed control system according to one aspect of the disclosed technology.

FIG. 6 is a chart showing predicted-actual glucose consumed and mean versus culture day.

FIG. 7 is a chart showing glucose measured and mean versus culture day.

FIG. 8 is a chart showing glucose measurement versus algorithm.

FIG. 9 is an example flow chart illustrating a process of controlling a nutrient feed in a cell culture process.

FIG. 10 is another example flow chart illustrating a process of controlling a glucose feed in a cell culture process.

FIG. 11 is a chart showing glucose measurement versus algorithm in a development (“Ambr250”) scale bioreactor, a pilot scale bioreactor, and a good manufacturing process (“GMP”) scale bioreactor for growing monoclonal antibody 1 (“MAB1”).

FIG. 12 is a is a chart showing glucose measured and mean versus culture day for MAB1.

FIG. 13 is a chart showing glucose measurement versus algorithm in a development scale (Ambr250) bioreactor, a pilot scale bioreactor, and a GMP scale bioreactor for growing bi-specific antibody 1 (“BsAb1”).

FIG. 14 is a is a chart showing glucose measured and mean versus culture day for BsAB1.

### DETAILED DESCRIPTION

Some implementations of the disclosed technology will be described more fully with reference to the accompanying drawings. This disclosed technology may, however, be embodied in many different forms and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods. Such other components not described herein may include, but are not limited to, for example, components developed after development of the disclosed technology.

It is also to be understood that the mention of one or more method steps does not preclude the presence of additional method steps or intervening method steps between those steps expressly identified.

It is to be noted, unless otherwise clear from the context, that the term “a” or “an” entity refers to one or more of that entity; for example, “an amino acid,” is understood to represent one or more proteins. As such, the terms “a” (or “an”), “one or more,” and “at least one” can be used interchangeably herein.

The term “nutrient” may refer to any compound, molecule, or substance used by an organism to live, grow, or otherwise add biomass. Examples of nutrients may include carbohydrate sources (e.g., simple sugars such as glucose, galactose, maltose or fructose, or more complex sugars), amino acids, vitamins (e.g., B group vitamins (e.g., B12), vitamin A vitamin E, riboflavin, thiamine and biotin). In the present invention, one or more nutrients may be utilized as a surrogate molecule to determine the amount of total nutrient media to add to a bioreactor. In some embodiments, the term “nutrient” may refer to simple sugars, vitamins, and amino acids.

The term “amino acid” may refer any of the twenty standard amino acids, i.e., glycine, alanine, valine, leucine, isoleucine, methionine, proline, phenylalanine, tryptophan, serine, threonine, asparagine, glutamine, tyrosine, cysteine, lysine, arginine, histidine, aspartic acid and glutamic acid, single stereoisomers thereof, and racemic mixtures thereof. The term “amino acid” can also refer to the known non-standard amino acids, e.g., 4-hydroxyproline, hydroxy-proline, s-sulfocysteine, phosphotyrosine, ε-N,N,N-trimethyllysine, 3-methylhistidine, 5-hydroxylysine, O-phosphoserine, γ-carboxyglutamate, ε-N-acetyllysine, ω-N-methylarginine, N-acetylserine, N,N,N-trimethylalanine, N-formylmethionine, γ-aminobutyric acid, histamine, dopamine, thyroxine, citrulline, ornithine, β-cyanoalanine, homocysteine, azaserine, and S-adenosylmethionine. In some embodiments, the amino acid is glutamate, glutamine, lysine, tyrosine or valine. In some embodiments, the amino acid is glutamate or glutamine.

The terms “nutrient media,” “feed media,” “feed,” “total feed,” and “total nutrient media” may be used interchangeably, and may include a “complete” media used to grow, propagate, and add biomass to a cell line. Nutrient media may be distinguished from a substance or simple media which by itself is not sufficient to grow and propagate a cell line. Thus, for example, glucose or simple sugars by themselves are not nutrient media, since in the absence of other required nutrients, they would not be sufficient to grow and propagate a cell line.

In one embodiment, the disclosed system includes an automated process with feedback control during the cell culture process.

In one embodiment, both lactate and glucose are measured, and step changes (0.5 g/L at a time) are made to the glucose target.

In one embodiment, only glucose is measured. By using only glucose that streamlines the process in the lab and makes it easier for the bioreactor operators to use the algorithm and feed glucose appropriately.

In one embodiment, glucose target is calculated once per day. The algorithm may be updated to allow for multiple measurements and the target being for the next 24 hrs.

The algorithm concept originated from taking the thought process human operators used and translating that to a flow diagram that used measured glucose and lactate to make step changes to increase or decrease the glucose target.

The disclosed glucose algorithm tends to be more accurate than a human operator because the human operator tends to underestimate glucose (leading to depletion events) or overestimate glucose targets to ensure that glucose is not depleted. The disclosed algorithm is better at controlling glucose at a desired level than a human operator likely could.

The disclosed method may be used for other monosaccharides, nutrients, etc. Non-limiting examples of such monosaccharides or nutrients include glutamate, galactose, lactate, and glutamine.

The disclosed technology may minimize glucose to avoid glycation.

In some embodiments, additional nutrient media may be added to the bioreactor cell culture in an amount sufficient to maintain a substantially stable concentration of the amino acid throughout a bioreactor process.

In some embodiments, the bioreactor cell culture may include Chinese Hamster Ovary (CHO) cells, HEK-293 cells, or VERO cells. In some embodiments, the bioproduct may be an antibody or antibody-like polypeptide.

In one embodiment, the methods of the present invention may be performed in the presence of any cell culture media. For example, the bioreactor process may be performed in the presence of serum-free media, protein-free media (including, but not limited to, protein-free media containing protein hydrolysates), or chemically defined media.

Various analytical devices may be used in the present invention. The analytical devices may include any instrument or process that can detect and/or quantify a surrogate molecule or marker, e.g., an amino acid or other substituents of cell culture media (e.g., a vitamin, a mineral, an ion, sugar, etc.). The analytical device may be an apparatus for performing gas chromatography, HPLC, cation exchange chromatography, anion exchange chromatography, size exclusion chromatography, an enzyme-catalyzed assay, and/or a chemical reaction assay.

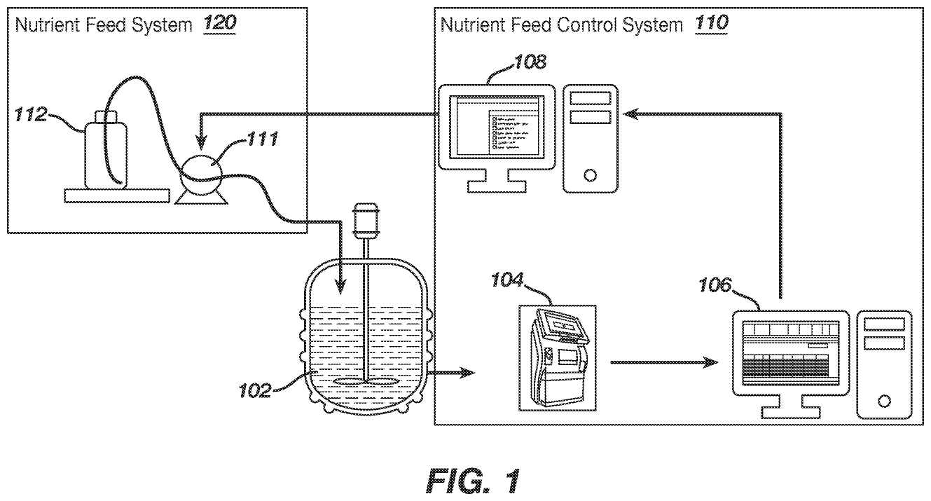

As shown in FIG. 1, a production reactor 102 may include mammalian cell culture. The production reactor 102 may be a bioreactor, a cell culture reactor or a sample bioreactor. The production reactor 102 may be at least one of the following: a well plate, a shake flask, a bench top vessel, and a commercial scale (e.g., 15 kL) stainless steel reactor. Reaction sample may be withdrawn from the production reactor 102, and sent to a nutrient feed control system 110. The nutrient feed control system 110 may include a glucose measurement system 104 that performs glucose measurement. The glucose measurement may be performed either offline or online. The nutrient feed control system 110 may also include a glucose target prediction system 106 that receives glucose measurement from the glucose measurement system 104, and performs glucose target prediction. The nutrient feed control system 110 may include a glucose calculation system 108 may then use the predicted glucose target to calculate an amount of glucose to add, and send an instruction to a nutrient feed system 120. In one embodiment, processes performed by the glucose measurement system 104, the glucose target prediction system 106 and the glucose calculation system 108 may be completed by one or more processors.

The nutrient feed system 120 may include a pump 111 which feeds the correct amount of glucose from glucose feed 112 to the production reactor 102.

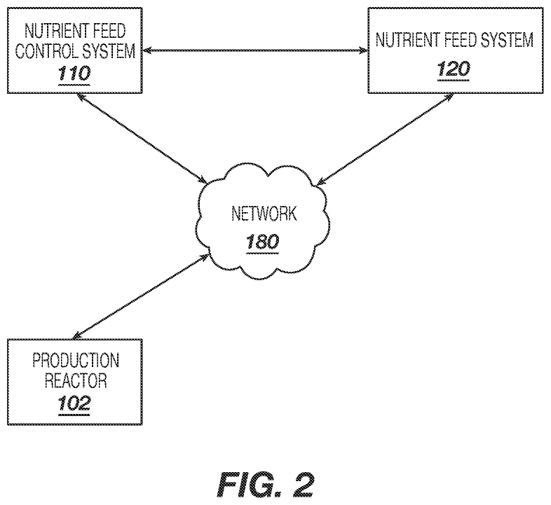

FIG. 2 illustrates schematic diagram of an example environment that may be used to implement one or more embodiments of the present disclosure. The nutrient feed control system 110 may communicate with the production reactor 102 and the nutrient feed system 120 over a network 180. The nutrient feed control system 110 may direct the nutrient feed system 120 to feed one or more nutrients to the production reactor 102.

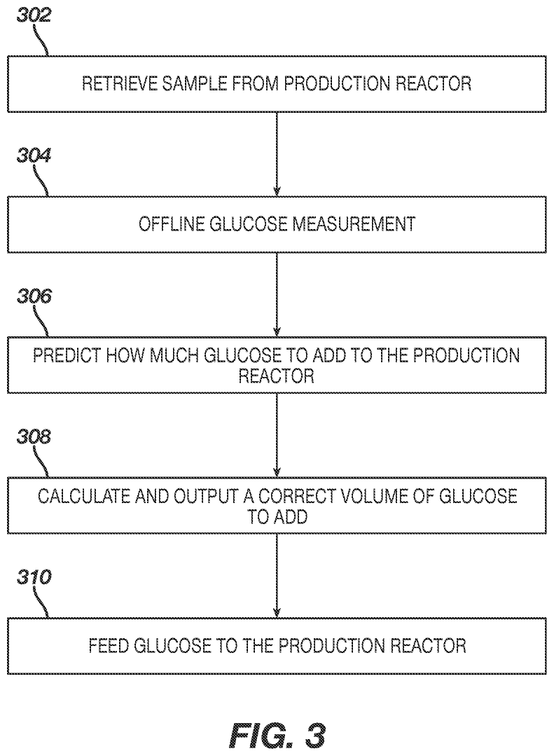

FIG. 3 illustrates a flow diagram for glucose algorithm. At 302, the production reactor 102 may provide sample. At 304, the glucose measurement system 104 may receive the sample, and conduct glucose measurement. At 306, the glucose target prediction system 106 may predict how much glucose to add to the production reactor 102. At 308, the glucose calculation system 108 may calculate and output a correct volume of glucose to add. At 310, the correct volume of glucose may be fed to the production reactor 102. This algorithm may be applicable to preculture. For example, the algorithm may be used to feed glucose to intensified seed trains. The algorithm may also be applicable to N−1 perfusion process and production perfusion processes.

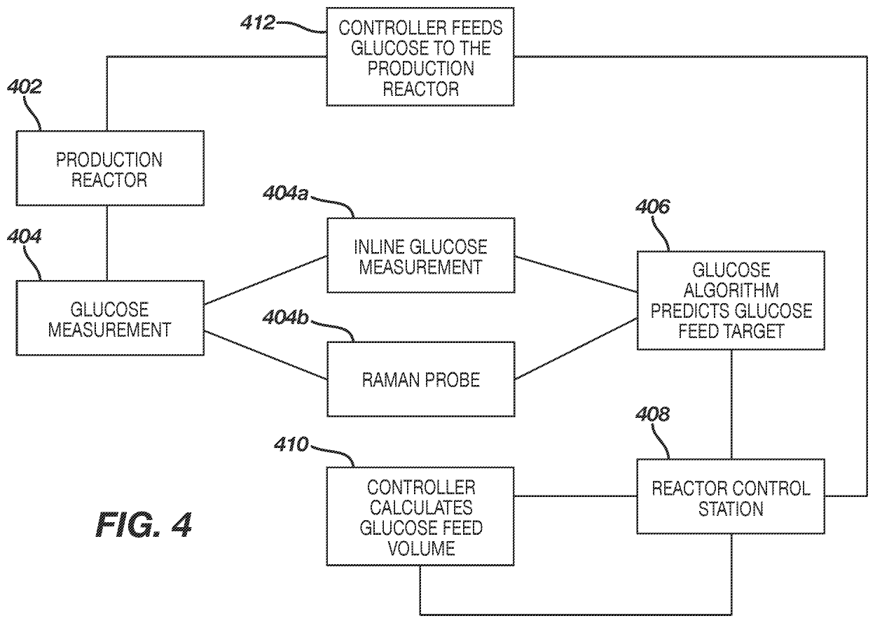

FIG. 4 illustrate an example of an automated process. At 402, the production reactor 102 may provide sample. At 404, glucose measurement may be conducted, such as inline glucose measurement (e.g., NovaFlex) at 404a, or Raman probe at 404b. An instrument may be used to measure offline pH as well as glucose and lactate. At 406, the nutrient feed control system 110 may perform predict glucose feed target. At 408, a reactor control station may process the predicted glucose feed target. At 410, a controller may calculate glucose feed volume. At 412, the controller may feed glucose to the production reactor 102.

The methods disclosed herein may increase production in subsequent bioreactor cell cultures. In some embodiments, the method may enhance the quantity of an antibody (or other bioproduct) produced, or decreasing antibody (or other bioproduct) production time, in a bioreactor cell culture producing the antibody (or other bioproduct). The method may include analyzing a culture sample (with or without extracting a sample from the bioreactor) by means of an automated sampling device (such as, for example, by means of off-line, on-line, in-line or at-line sample analysis). The method may include analyzing a culture sample (e.g., the concentration of residual glucose) by means of an automated analytical device to generate data representative of the quantity of a nutrient (or other surrogate marker). The method may include processing the generated data (e.g., from assaying residual glucose from the sample) by means of an algorithm or computer-based processing program wherein the processed data is used to determine an amount of additional nutrient media to add to the bioreactor. The method may include adding the determined amount of nutrient media determined to the bioreactor by means of an automated feed device. The method may include recording the time and amount of each nutrient media addition.

Mammalian cells may include any mammalian cells that are capable of growing in culture. Exemplary mammalian cells include, e.g., CHO cells (including CHO-K1, CHOKISV®, CHO DUKX-B11, CHO DG44), VERO, BHK, HeLa, CVI (including Cos; Cos-7), MDCK, 293, 3T3, C127, myeloma cell lines (especially murine), PC12, HEK-293 cells (including HEK-293T and HEK-293E), PER C6, Sp2/0, NSO and W138 cells. Mammalian cells derived from any of the foregoing cells may also be used. In some embodiments, the bioreactor cell culture may comprise Chinese Hamster Ovary (CHO) cells, HEK-293 cells, or VERO cells.

The steps of the disclosed method may be repeated, and may occur at various intervals. In some embodiments, steps disclosed herein may be repeated greater than 10 times throughout a bioreactor process, or 10 to 1000 times, 20 to 500 times or 30 to 100 times throughout a bioreactor process. In some embodiments, steps may be repeated about every 4 minutes, 10 minutes, 30 minutes, 60 minutes, 2 hours, 3 hours, 6 hours, 8 hours, 12 hour, 16 hours, 18 hours, or 24 hours throughout a bioreactor process, or about every 4 to 18 hours, or about every 10 minutes to about every 6 hours throughout a bioreactor process. In specific embodiments, the method comprises measuring the amount of residual nutrient (e.g., residual glucose) once per day, with a target concentration of the nutrient (e.g., glucose) generated for a period about one day, or about 24 hours later. In certain embodiments, the method comprises measuring the amount of residual nutrient (e.g., residual glucose) multiple times per day, e.g., twice, three times, or four times per day, with a target concentration of the nutrient (e.g., glucose) generated for a period about 24 hours the measurement.

The steps of the methods disclosed herein may occur in a relatively short amount of time, i.e., the sampling, analysis, and addition of additional nutrient media can occur relatively quickly. In some embodiments, steps of the disclosed method are performed within about 1 minute to about 2 hours.

In some embodiments, steps of the disclosed method are performed by one or more automated devices. The terms “automatic”, “automatically”, or “automated” describe one or more mechanical devices that perform one or more tasks without any human intervention or action, except for any human intervention or action necessary to initially prepare the device or devices for task performance; or as may be required to maintain automatic operation of the device or devices. A “mechanical device” that performs one or more tasks automatically may, optionally, include a computer and the necessary instructions (code) therein to process collected data which may be used therein for decision making purposes to control and direct performance of the device or devices, such as in controlling the timing, duration, frequency, kind, and/or character of tasks to be performed.

In various embodiments, “off-line” analysis refers to permanently removing a sample from the production process and analyzing the sample at a later point in time such that the data analysis does not convey real-time or near real-time information about in-process conditions. In some embodiments, one or more analytical devices are used off-line.

In one embodiment, an analytical device (or a sensor-portion connected thereto) may be introduced directly into a bioreactor or purification unit, or the device or sensor-portion may be separated from the bioreactor or purification unit by an appropriate barrier or membrane.

In some embodiments, the analytical device may be a kit, e.g., a test strip, which can be placed in contact with the sample to give rapid determination of the cellular concentration. In some embodiment, the kit may comprise a substrate which produces a chemical and/or enzyme-linked reaction to produce a detectable signal in the presence of a surrogate marker, or a specific concentration of a surrogate marker. The detectable signal may include, e.g., a colormetric change or other visual signal. In some embodiments, the analytical device may be a disposable analytical device, e.g., a disposable test strip. Such kits may be useful due to their ease of operation and their reduced costs relative to other larger, more complicated analytical devices. Such kits may also be useful during small scale cell culture propagation to determine that optimal health and productivity of the culture.

In various embodiments, the glucose algorithms and methods described herein may be effective to achieve a residual glucose level at one day post-feeding of between 0 to 3 g/L, 0.5 to 2 g/L, 2 to 5 g/L, less than 1 g/L, or less than 2 g/L.

Various bioproducts may be envisioned in the present invention. In some embodiments, the bioproduct may be an antibody, recombinant protein, glycoprotein, or fusion protein. In some embodiments, the bioproduct may be a soluble protein. In some embodiments, the bioproduct may be an antibody, antibody fragment or modified antibody (e.g., a multivalent antibody, a domain-deleted antibody, a multimeric antibody, a hinge-modified antibody, a stabilized antibody, a multispecific antibody, a linear antibody, an scFv, a linked ScFv antibody, a multivalent linear antibody, a multivalent antibody without Fc, a Fab, a multivalent Fab, etc.).

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

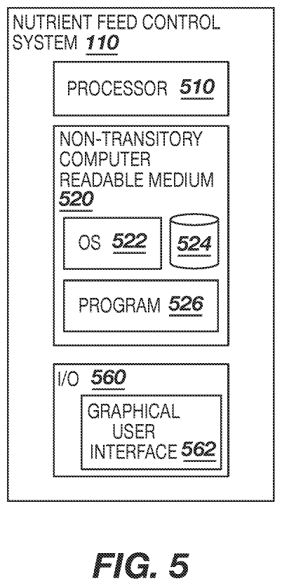

FIG. 5 is a block diagram of the nutrient feed control system 110 according to one aspect of the disclosed technology. The nutrient feed control system 110 may include one or more processors 510. The processes performed by the glucose measurement system 104, the glucose target prediction system 106 and the glucose calculation system 108 may be completed by one or more processors 510.

Referring to FIG. 5, a processor 510 may include one or more of a microprocessor, microcontroller, digital signal processor, co-processor or the like or combinations thereof capable of executing stored instructions and operating upon stored data. The processor 510 may be one or more known processing devices, such as a microprocessor from the Pentium™ family manufactured by Intel™ or the Turion™ family manufactured by AMD™. The processor 510 may constitute a single core or multiple core processor that executes parallel processes simultaneously. For example, the processor 510 may be a single core processor that is configured with virtual processing technologies. In certain embodiments, the processor 510 may use logical processors to simultaneously execute and control multiple processes. The processor 510 may implement virtual machine technologies, or other similar known technologies to provide the ability to execute, control, run, manipulate, store, etc. multiple software processes, applications, programs, etc. One of ordinary skill in the art would understand that other types of processor arrangements could be implemented that provide for the capabilities disclosed herein.

A non-transitory computer readable medium 520 may include, in some implementations, one or more suitable types of memory (e.g., such as volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like), for storing files including an operating system 522, application programs (including, for example, a web browser application, a widget or gadget engine, and or other applications, as necessary), executable instructions and data. In one embodiment, the processing techniques described herein are implemented as a combination of executable instructions and data within the non-transitory computer readable medium 520. The non-transitory computer readable medium 520 may include one or more memory devices that store data and instructions used to perform one or more features of the disclosed embodiments. The non-transitory computer readable medium 520 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational or non-relational databases. The non-transitory computer readable medium 520 may include software components that, when executed by the processor 510, perform one or more processes consistent with the disclosed embodiments. In some embodiments, the non-transitory computer readable medium 520 may include a database 524 to perform one or more of the processes and functionalities associated with the disclosed embodiments. The non-transitory computer readable medium 520 may include one or more programs 526 to perform one or more functions of the disclosed embodiments. Moreover, the processor 510 may execute one or more programs 526 located remotely from the system 110. For example, the system 110 may access one or more remote programs 526, that, when executed, perform functions related to disclosed embodiments.

The system 110 may also include one or more I/O devices 560 that may comprise one or more interfaces for receiving signals or input from devices and providing signals or output to one or more devices that allow data to be received and/or transmitted by the system 110. For example, the system 110 may include interface components, which may provide interfaces to one or more input devices, such as one or more keyboards, mouse devices, touch screens, track pads, trackballs, scroll wheels, digital cameras, microphones, sensors, and the like, that enable the system 110 to receive data from one or more users. The system 110 may include a display, a screen, a touchpad, or the like for displaying images, videos, data, or other information. The I/O devices 560 may include the graphical user interface 562.

In exemplary embodiments of the disclosed technology, the system 110 may include any number of hardware and/or software applications that are executed to facilitate any of the operations. The one or more I/O interfaces 560 may be utilized to receive or collect data and/or user instructions from a wide variety of input devices. Received data may be processed by one or more computer processors as desired in various implementations of the disclosed technology and/or stored in one or more memory devices.

The networks 180 may include a network of interconnected computing devices more commonly referred to as the internet. The network 180 may be of any suitable type, including individual connections via the internet such as cellular or WiFi networks. In some embodiments, the network 180 may connect terminals, services, and mobile devices using direct connections such as radio-frequency identification (RFID), near-field communication (NFC), Bluetooth™, low-energy Bluetooth™ (BLE), WiFi™, ZigBee™, ambient backscatter communications (ABC) protocols, USB, WAN, or LAN. Because the information transmitted may be personal or confidential, security concerns may dictate one or more of these types of connections be encrypted or otherwise secured. In some embodiments, however, the information being transmitted may be less personal, and therefore the network connections may be selected for convenience over security. The network 180 may comprise any type of computer networking arrangement used to exchange data. For example, the network 180 may be the Internet, a private data network, virtual private network using a public network, and/or other suitable connection(s) that enables components in system environment to send and receive information between the components of system 100. The network 180 may also include a public switched telephone network (“PSTN”) and/or a wireless network. The network 180 may also include local network that comprises any type of computer networking arrangement used to exchange data in a localized area, such as WiFi, Bluetooth™ Ethernet, and other suitable network connections that enable components of system environment to interact with one another.

The processor 510 may implement a predictive cell-based glucose algorithm to estimate daily glucose target requirements. The processor 510 may calculate a daily glucose target for each bioreactor on each culture day. To predict the glucose requirements needed for the next day, the algorithm may make a key assumption: the cellular growth rate and the glucose consumption rate over the previous day remains the same for the following day. In making these assumptions, the next day's viable cell density (VCD) may be predicted based on the current day's VCD measurement and the growth rate from the previous day. The IVCD for the next day may be predicted, and the expected total daily glucose consumption may be calculated accordingly.

The presently disclosed algorithm may not be based on any historical database. By using the presently disclosed algorithm described herein, the glucose levels may be maintained between 1.5-3.5 g/L.

### 1. Cell Growth Calculations

Equations 1-3 show how data from the current and previous days may be leveraged to anticipate culture growth over the next day. The assumption underpinning this predicted IVCD is that growth rate does not change appreciably over the course of just one day. IVCD may be calculated using a mathematical average approach.

For culture day 0, growth rate, predicted VCD, and predicted IVCD may not be calculated. Equation 1 may be used to determine growth rate for all other culture days. Equations 2 and 3 show how the growth rate from the current day may be used to predict the VCD and IVCD for the following day.

$${GR}_{n} = ln ( \frac{{VCD}_{n}}{{VCD}_{n - 1}} ) \qquad [ 1 ]$$

$$predicted {VCD}_{n + 1} = {VCD}_{n} * exp ( {GR}_{n} ) \qquad [ 2 ]$$

$$predicted {IVCD}_{n + 1} = \frac{( predicted {VCD}_{n + 1} + {VCD}_{n} )}{2} + {IVCD}_{n} \qquad [ 3 ]$$

### 2. Glucose Consumption Calculations

Once calculated, the predicted IVCD may be applied to glucose consumption calculations to estimate the amount of glucose required for the next day. Equation 4 may calculate the daily specific glucose consumption rate over the previous day, using the IVCD method for specific metabolic rates. Next, the predicted daily glucose to be consumed may be calculated as the product of the specific glucose consumption rate from the current day and the difference of the predicted IVCD for the next day and the current IVCD, as illustrated in Equation 5. Finally, the glucose target (GTₙ) may be taken as the sum of the predicted daily glucose to be consumed (GCₙ₊₁) and an empirically determined glucose to maintain value (GMₙ₊₁), as illustrated in Equation 6.

$${q}_{gluc , n} = \frac{{GC}_{n}}{{IVCD}_{n} - {IVCD}_{n - 1}} \qquad [ 4 ]$$

$$predicted {GC}_{n + 1} = {q}_{gluc , n} * ( predicted {IVCD}_{n + 1} - {IVCD}_{n} ) \qquad [ 5 ]$$

$${GT}_{n} = predicted {GC}_{n + 1} + {GM}_{n + 1} \qquad [ 6 ]$$

In standard practice, GM may be set at 2 g/L for each culture day, but project teams may adjust this value as deemed necessary in subsequent bioreactor runs to prevent depletion or accumulation of glucose in the culture.

### 3. Verification

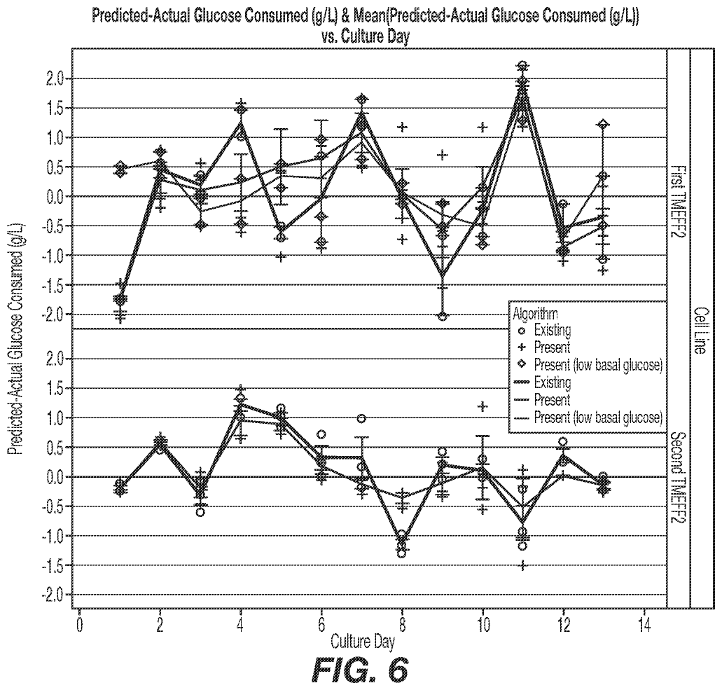

The glucose algorithm disclosed herein has been evaluated in two different recombinant cell lines: a first host cell line and a second host cell line. These groups are chosen to ensure the accuracy of the algorithm for different host cell lines and process parameters (e.g. target seeding density, glucose concentration in basal media). For example, the first cell line process bioreactors are seeded at 0.5×10⁶ vc/mL in basal media containing 4.2 g/L glucose, while the second cell line bioreactors are inoculated with a target seeding density of 1.5×10⁶ vc/mL in basal media containing 12 g/L glucose (except where otherwise indicated). Below, Table 1 lists relevant details about the bioreactor runs where the presently disclosed algorithm is implemented and compared against an existing glucose algorithm. Although 6 bioreactor runs using the recombinant cell line are fed using the presently disclosed algorithm, 4 of those reactors used basal media containing 12 g/L glucose, and the other 2 reactors used basal media containing 4.2 g/L glucose. This distinction is highlighted in FIGS. 6-8 described below. Each of those 6 reactors are fed using the glucose algorithm disclosed herein.

| | | | | # Reactors fed |
|---|---|---|---|---|
| Host | Cell | | # Reactors fed | using the |
| Cell | Line | Bioreactor | using existing | presently disclosed |
| Line | Number | Scale | glucose algorithm | glucose algorithm |
| First | C3464A | Ambr250 | 2 | 6 |
| Second | C3472B | 5L | 3 | 3 |

For each bioreactor run, glucose targets are calculated each day, using either the existing glucose algorithm or the presently disclosed glucose algorithm. VCD data and glucose measurements from the bioreactor runs are input into the glucose algorithm each day, and the predicted glucose consumption and glucose targets are calculated for each bioreactor and culture day. After the runs are complete, the actual amount of glucose consumed for each bioreactor and culture day are calculated. The actual amount of glucose consumed is subtracted from the predicted amount of glucose consumed to calculate the error of predicted glucose consumption for each bioreactor and culture day. Below, the individual data and summary statistics (mean and standard error) are reported for each culture day in FIG. 6. FIG. 6 illustrates that the actual glucose consumed is subtracted from the predicted glucose consumed for each culture day to assess the ability of the glucose algorithm to predict glucose consumption for the next day. Arrows indicate the day when glucose feed is first added to the culture. The mean difference for the presently disclosed algorithm is between median residual glucose values for the predictive cell-based glucose algorithm are between −0.86 and 1.67 g/L as compared to −1.35 and 1.94 g/L for the existing glucose algorithm. The means for all culture days across each cell line and glucose algorithm are summarized and tabulated below in Table 1.

TABLE 1
Predicted-Actual Glucose Consumed Data Summary

| Cell | | Mean Predicted-Actual |
|---|---|---|
| Line | Algorithm | Glucose Consumed |
| First | Existing algorithm | −1.35 to 1.94 g/L |
| First | Present algorithm (low basal glucose) | −0.75 to 1.62 g/L |
| First | Present algorithm | −0.86 to 1.67 g/L |
| Second | Existing algorithm | −1.20 to 1.22 g/L |
| Second | Present algorithm | −0.52 to 0.93 g/L |

For both cell lines, the present algorithm results in more accurate predictions for glucose consumption. Additionally, the present algorithm shows higher predicted-actual glucose consumed, in comparison to the existing glucose algorithm. As a result, the present glucose algorithm is less likely to underestimate glucose needs for the culture, compared to the existing glucose algorithm.

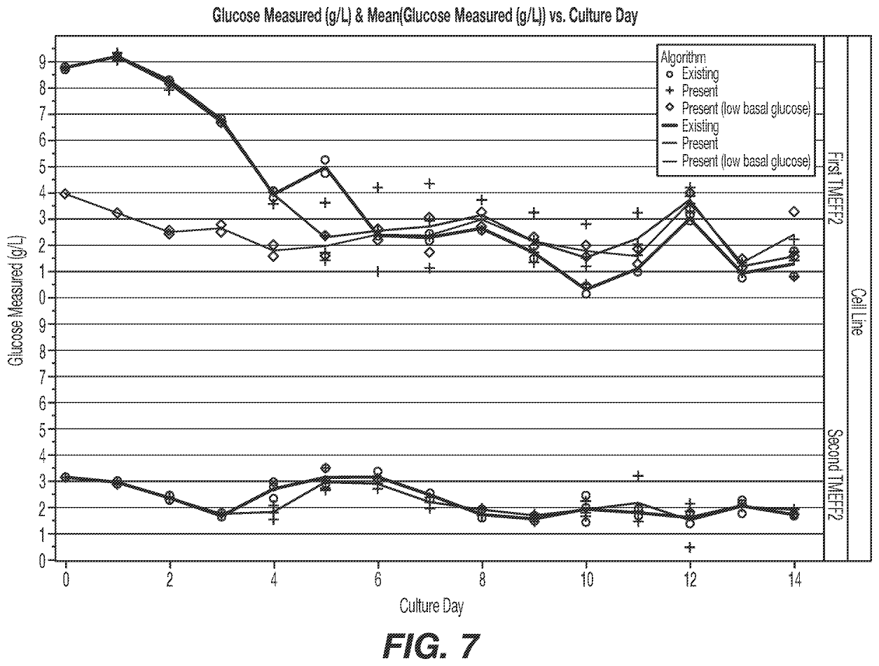

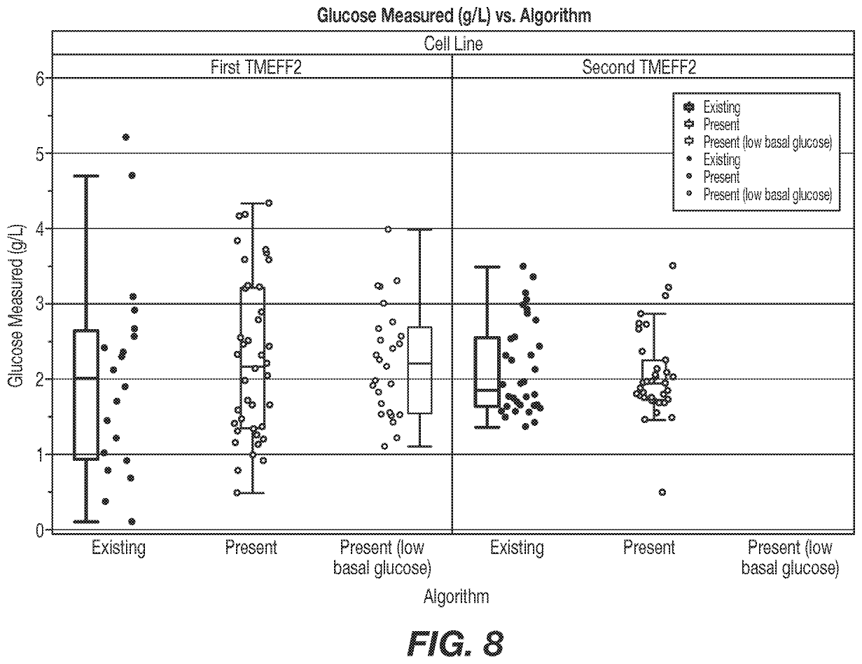

The in-process glucose measurements are also reported below in FIGS. 7-8, which demonstrate that the presently disclosed glucose algorithm does not result in extreme depletion or accumulation during the fed-batch process.

Referring to FIG. 7, daily glucose is measured to evaluate the ability of the presently disclosed glucose algorithm to control glucose within a desirable range of 1-3 g/L. The presently disclosed glucose algorithm appears to control glucose adequately, and similarly to the existing glucose algorithm throughout the run. The glucose values on Days 11 and 12 for M20K069 are not considered due to an unintentional overfeed on Day 10 by the operator. Additionally, reactors M20K196-M20K206 had inaccurate cell counts that likely affected glucose levels on Day 5. The arrows in FIG. 7 indicate the day that glucose feed is first required and added in the process.

Referring to FIG. 8, aggregated glucose measurements across culture days where the algorithm called for glucose feed on the previous day show that most glucose daily measurements maintain in the desired 1-3 g/L range. As a result, the presently disclosed glucose algorithm may reliably maintain glucose levels at the desired level throughout the process.

The desired range for glucose concentration 24 hours after sampling and feeding is 1-3 g/L. The presently disclosed glucose algorithm adequately controls glucose levels, with most data falling in the 1-3 g/L range. The middle 50% of data also falls within this range for all conditions with the exception of the present glucose algorithm with the first recombinant cell line, where the middle 50% of data were between 1.33 and 3.21 g/L. Additionally, the presently disclosed glucose algorithm maintains tighter control (a narrower range) of glucose levels, aside from the discrepancy with second reactor M20K069 mentioned in FIG. 7.

Slightly higher glucose values are maintained using the first recombinant cell line with the presently disclosed glucose algorithm, which means there is reduced risk of glucose depletion. This is likely because the database used to build the historical algorithm is based on bioreactor runs with target seeding density of 0.5×10⁶ vc/mL according to the existing algorithm, as compared to the target seeding density of 1.5×10⁶ vc/mL according to the presently disclosed algorithm. This highlights a key advantage of utilizing the presently disclosed glucose algorithm. Specifically, the predictive nature of the model circumvents the need to amass a large database of daily glucose consumption prior to implementation of the algorithm.

In view of the above, the presently disclosed glucose algorithm may reliably predict daily total glucose consumption and maintain glucose levels in a desirable range ˜1-3 g/L. As the presently disclosed glucose algorithm is implemented, more data may be aggregated with this database to verify these trends. Once glucose targets have been generated and tested using the algorithm, project teams may choose to adjust the targets by increasing or decreasing the residual glucose to maintain value. If deemed necessary, targets may be adjusted based on measured daily glucose consumption in the project's target process.

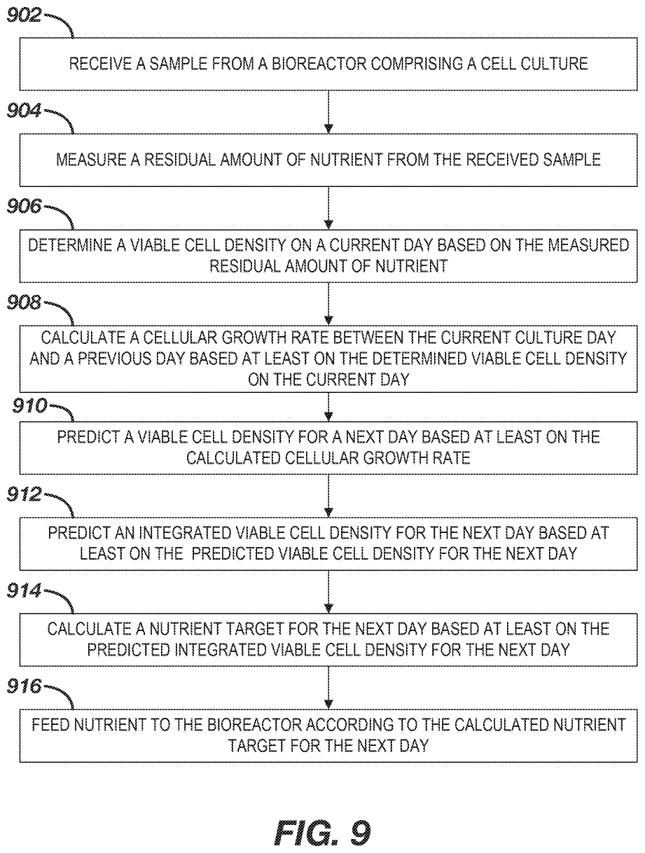

FIG. 9 is an example flow chart illustrating a process of controlling a nutrient feed in a cell culture process. At 902, a sample may be received from a bioreactor comprising a cell culture. At 904, a residual amount of nutrient may be measured from the received sample. At 906, the processor 510 may determine variable cell density on a current day based on the measured residual amount of nutrient. At 908, the processor 510 may calculate a cellular growth rate between the current culture day and a previous day based at least on the determined viable cell density on the current day. At 910, the processor 510 may predict a viable cell density for a next day based at least on the calculated cellular growth rate. At 912, the processor 510 may predict an integrated viable cell density for the next day based at least on the predicted viable cell density for the next day. At 914, the processor 510 may calculate a nutrient target for the next day based at least on the predicted integrated viable cell density for the next day. At 916, nutrient may be fed to the bioreactor according to the calculated nutrient target for the next day.

In one embodiment, the processor 510 may calculate the cellular growth rate between the current culture day and a previous day based on the determined viable cell density on the current day, and a viable cell density measured on a previous day.

In one embodiment, the processor 510 may predict the viable cell density for the next day based on the calculated cellular growth rate and the determined viable cell density on the current day.

In one embodiment, the processor 510 may predict the integrated viable cell density for the next day based on the predicted viable cell density for the next day, the determined viable cell density on the current day, and an integrated viable cell density for the current day.

In one embodiment, the processor 510 may calculate a daily specific nutrient consumption rate over the previous day based at least on the calculated cellular growth rate. The processor 510 may predict an amount of nutrient to be consumed between the current day and the next day based on the daily specific nutrient consumption rate. The processor 510 may calculate the nutrient target based at least on the predicted amount of nutrient to be consumed.

In one embodiment, the processor 510 may calculate the daily specific nutrient consumption rate over the previous day based on the calculated cellular growth rate, an integrated viable cell density for the current day, and an integrated viable cell density for the previous day.

In one embodiment, the processor 510 may predict the amount of nutrient to be consumed between the current day and the next day based on the daily specific nutrient consumption rate, the predict integrated viable cell density for the next day, and an integrated viable cell density for the current day.

In one embodiment, the processor 510 may calculate the nutrient target based on the predicted nutrient to be consumed and an empirically determined nutrient to maintain value.

In one embodiment, the non-transitory computer readable medium 520 may store one or more of the following: the viable cell density measured on the previous day, the integrated viable cell density for the current day, the integrated viable cell density for the previous day, and the empirically determined nutrient to maintain value, which may be retrieved from the non-transitory computer readable medium 520.

In one embodiment, the nutrient may be selected from glucose, glutamate, galactose, lactate, and glutamine.

In one embodiment, the nutrient may include one or more monosaccharides.

In one embodiment, the residual nutrient measurement may include assaying a nutrient concentration in the bioreactor.

In one embodiment, the residual nutrient measurement may include performing one or more of offline nutrient measurement and inline nutrient measurement.

In one embodiment, the residual nutrient measurement may be performed by one or more of the following: a NovaFlex device and a Raman Probe.

In one embodiment, the bioreactor may be one or more of the following: a Chinese hamster ovary (CHO) cell bioreactor, and a 5 L bioreactor. Other mammalian cell types that may be used in manufacturing biologics besides CHO, including recombinant cells and the like. Non-limiting examples of such mammalian cell types include HEK, 293 and PerC6. This process may be also used for other non-mammalian cell types, such as, for example, yeast and bacteria.

In one embodiment, cells in the bioreactor may be mammalian cells.

In one embodiment, the cells may be CHO cells.

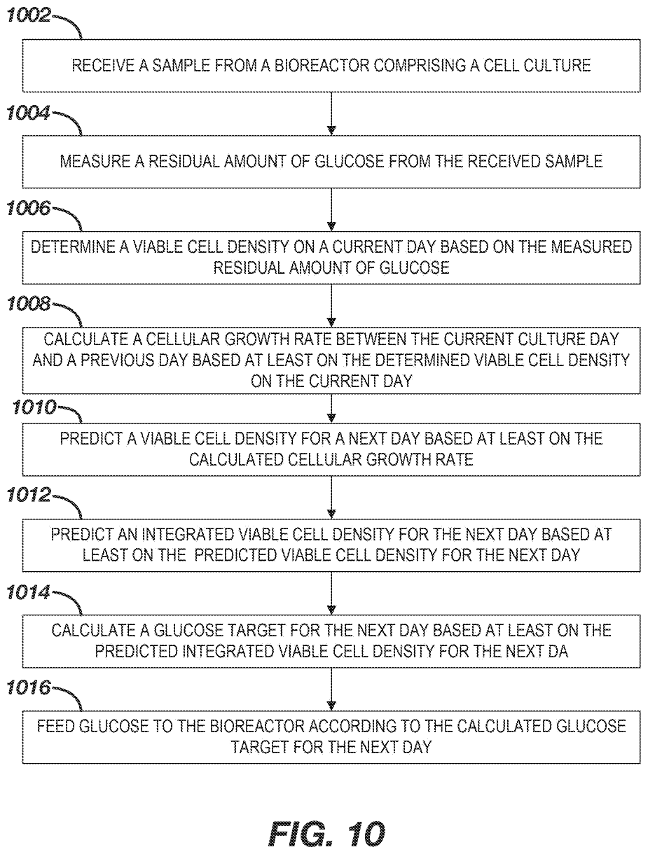

FIG. 10 is another example flow chart illustrating a process of controlling a glucose feed in a cell culture process. At 1002, a sample may be received from a bioreactor comprising a cell culture. At 1004, a residual amount of glucose may be measured from the received sample. At 1006, the processor 510 may determine variable cell density on a current day based on the measured residual amount of glucose. At 1008, the processor 510 may calculate a cellular growth rate between the current culture day and a previous day based at least on the determined viable cell density on the current day. At 1010, the processor 510 may predict a viable cell density for a next day based at least on the calculated cellular growth rate. At 1012, the processor 510 may predict an integrated viable cell density for the next day based at least on the predicted viable cell density for the next day. At 1014, the processor 510 may calculate a glucose target for the next day based at least on the predicted integrated viable cell density for the next day. At 1016, glucose may be fed to the bioreactor according to the calculated glucose target for the next day.

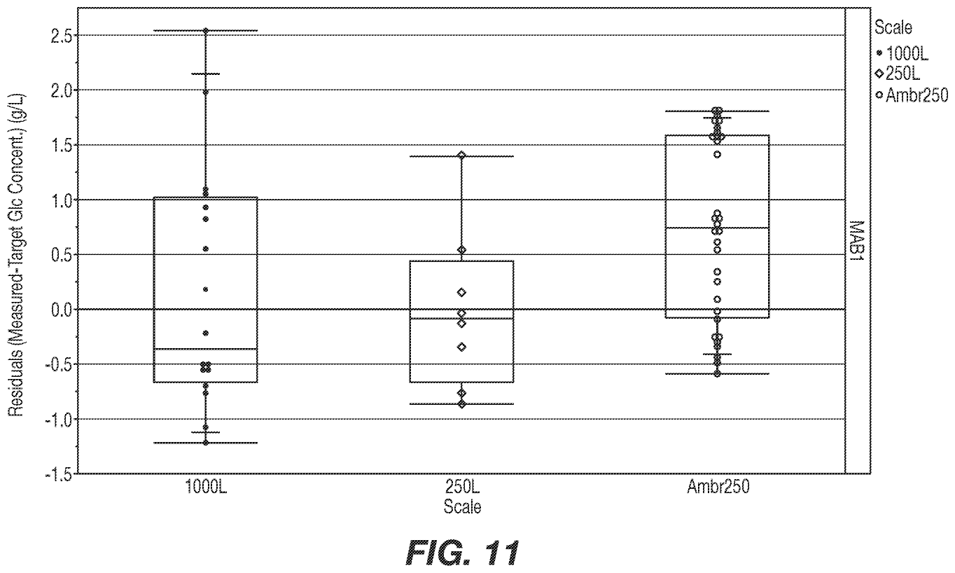

In another example, FIG. 11 is a chart showing glucose measurement versus algorithm in a development scale bioreactor, a pilot scale bioreactor, and a GMP scale bioreactor for growing MAB1. The daily glucose targets developed for the GMP bioreactor process for MAB1 were predicted during a pilot (Ambr250) experiment using the present algorithm. The present algorithm provided daily glucose feed targets such that the residual glucose levels as measured on the following culture day would be 2 g/L. FIG. 11 shows the median difference between control conditions within the experimental design and a residual glucose concentration of 2 g/L as 0.75 g/L for the pilot (Ambr250) experiment. The predicted glucose targets were then used to control the glucose between 1 g/L and 3 g/L for the pilot scale bioreactor experiment (250 L scale) and the GMP scale bioreactor (1000 L scale).

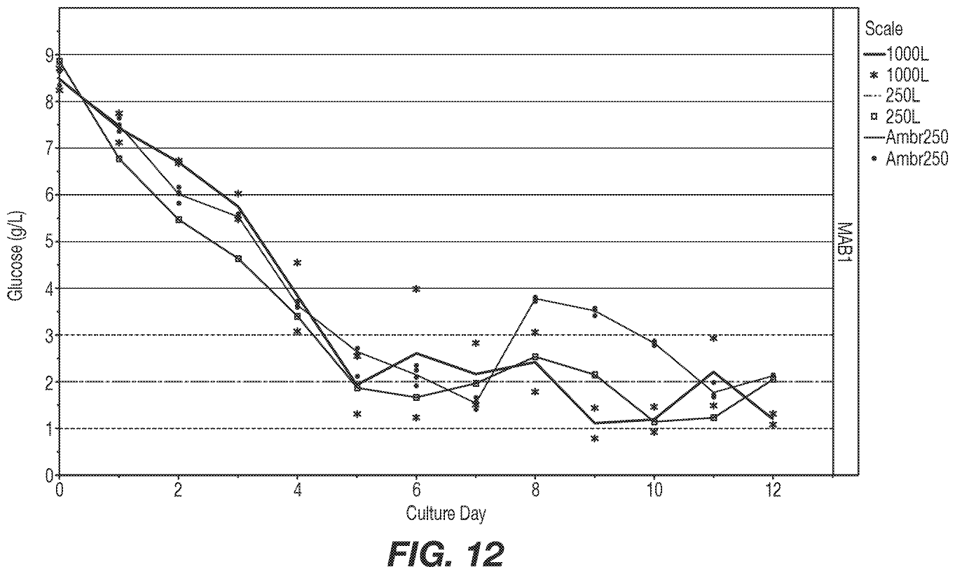

As shown in FIG. 12, both the development scale and the GMP scale processes can use the same targets that were identified in the pilot (Ambr250) experiment for growing MAB1. In some examples, day seven and day eight targets can be reduced by 1.5 g/L each for the development scale and GMP scale bioreactor processes as compared to the pilot experiment in order to bring the glucose concentration within the 1-3 g/L control range for each bioreactor process. FIG. 12 shows that by using the present algorithm, glucose was well controlled within the desired range for each bioreactor process. In FIG. 12, the dashed reference lines represent target control regions for glucose (e.g., 1-3 g/L) to minimize product glycation. The lines of FIG. 12 represent averages of multiple bioreactor runs.

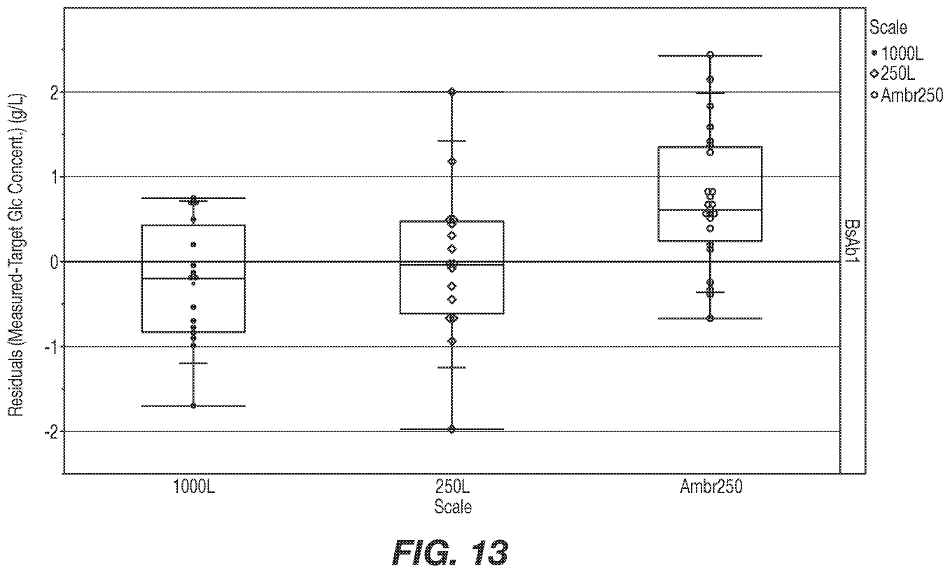

In another example, daily glucose targets were developed for the production bioreactor process for BsAb1 using the present algorithm, as shown in FIG. 13. The daily glucose targets were predicted using the pilot (Ambr250) experiment. The present algorithm provided daily glucose feed targets such that the residual glucose levels as measured on the following culture day would be 2 g/L. FIG. 13 shows the median difference between control conditions within the experimental design and a residual glucose concentration of 2 g/L as 0.62 g/L for the pilot (Ambr250) experiment. The predicted glucose targets were then used to control the glucose between 1 g/L and 3 g/L for the pilot scale bioreactor experiment (250 L scale) and the GMP scale bioreactor (1000 L scale).

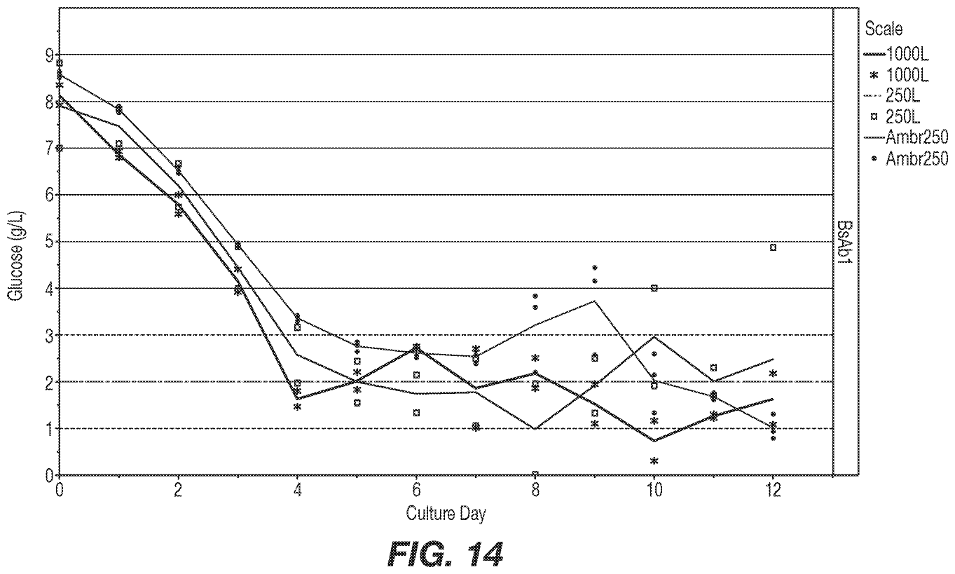

As shown in FIG. 14, both the development scale and the GMP scale processes can use the same targets that were identified in the pilot (Ambr250) experiment for growing BsAb1. In some examples, small modifications (e.g., less than 1 g/L) were made to the daily glucose targets predicted by the present algorithm to fine tune the results for the development scale and the GMP scale processes in order to keep the glucose concentration within the 1-3 g/L control range. FIG. 14 shows that by using the present algorithm, glucose was well controlled within the desired range for each bioreactor process. In FIG. 14, the dashed reference lines represent target control regions for glucose (e.g., 1-3 g/L) to minimize product glycation. The lines of FIG. 14 represent averages of multiple bioreactor runs.

The results of utilizing the present algorithm for controlling glucose feed of MAB1 and BsAb1 shown in FIGS. 11-14 show that the present algorithm can be effect to provide glucose targets that are robust and transferrable across various bioreactor scales, such as the pilot (Ambr250) scale, the developmental (250 L) scale, and the GMP (1000 L) scale.

Below is a list of abbreviations and definitions.

BioTD API may refer to biotherapeutics development active pharmaceutical ingredient.

BioTD CDS may refer to biotherapeutics development cell and developability sciences.

ELN may refer to electronic laboratory notebook.

GCₙ may refer to glucose consumed (g/L) between the current day and the day prior.

GCₙ₊₁ may refer to glucose to be consumed (g/L) between the current day and the next day.

GMₙ₌₁ may refer to glucose to maintain (g/L) for the next day.

GRₙ may refer to cellular growth rate (day⁻¹) calculated between the current day and the previous day.

GTₙ may refer to glucose target (g/L) for current day.

HT may refer to high titer.

IQR may refer to interquartile range.

IVCDₙ may refer to integrated viable cell density (cells/mL*day) calculated on the current day.

IVCDₙ₋₁ may refer to integrated viable cell density (cells/mL*day) calculated on the previous day.

IVCDₙ₊₁ may refer to integrated viable cell density (cells/mL*day) calculated for the next day.

q_{glc.n} may refer to specific glucose consumption rate (mg/(cell*day)) for the current day.

VCDₙ may refer to viable cell density (cells/mL) measured on the current culture day.

VCDₙ₋₁ may refer to viable cell density (cells/mL) measured on the previous culture day.

VCDₙ₊₁ may refer to viable cell density (cells/mL) for the next culture day.

MAB1 may refer to monoclonal antibody 1.

BsAB1 may refer to bi-specific antibody 1.

GMP may refer to good manufacturing process.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

Certain implementations of the disclosed technology are described above with reference to block and flow diagrams of systems and methods and/or computer program products according to example implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, or may not necessarily need to be performed at all, according to some implementations of the disclosed technology.

These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

Implementations of the disclosed technology may provide for a computer program product, comprising a computer-usable medium having a computer-readable program code or program instructions embodied therein, said computer-readable program code adapted to be executed to implement one or more functions specified in the flow diagram block or blocks. The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

1. A method of controlling a nutrient feed in a cell culture process, comprising:
receiving a sample from a bioreactor comprising a cell culture;
measuring a residual amount of nutrient from the received sample;
determining a viable cell density on a current day based on the measured residual amount of nutrient;
calculating a cellular growth rate between the current culture day and a previous day based at least on the determined viable cell density on the current day;
predicting a viable cell density for a next day based at least on the calculated cellular growth rate;
predicting an integrated viable cell density for the next day based at least on the predicted viable cell density for the next day;
calculating a nutrient target for the next day based at least on the predicted integrated viable cell density for the next day; and
feeding nutrient to the bioreactor according to the calculated nutrient target for the next day.

2. The method of claim 1, wherein the cellular growth rate is calculated between the current culture day and a previous day based on the determined viable cell density on the current day, and a viable cell density measured on a previous day.

3. The method of claim 2, wherein the viable cell density measured on the previous day is retrieved from a non-transitory storage medium.

4. The method of any one of claims 1 to 3, wherein the viable cell density for the next day is predicted based on the calculated cellular growth rate and the determined viable cell density on the current day.

5. The method of any one of claims 1 to 4, wherein the integrated viable cell density for the next day is predicted based on the predicted viable cell density for the next day, the determined viable cell density on the current day, and an integrated viable cell density for the current day.

6. The method of claim 5, wherein the integrated viable cell density for the current day is retrieved from a non-transitory storage medium.

7. The method of any one of claims 1 to 6, further comprising:
calculating a daily specific nutrient consumption rate over the previous day based at least on the calculated cellular growth rate,
predicting an amount of nutrient to be consumed between the current day and the next day based on the daily specific nutrient consumption rate, and
calculating the nutrient target based at least on the predicted amount of nutrient to be consumed.

8. The method of claim 7, wherein the daily specific nutrient consumption rate over the previous day is calculated based on the calculated cellular growth rate, an integrated viable cell density for the current day, and an integrated viable cell density for the previous day.

9. The method of claim 8, wherein the integrated viable cell density for the current day and the integrated viable cell density for the previous day are retrieved from a non-transitory storage medium.

10. The method of claim 7, wherein the amount of nutrient to be consumed between the current day and the next day is predicted based on the daily specific nutrient consumption rate, the predict integrated viable cell density for the next day, and an integrated viable cell density for the current day.

11. The method of claim 7, wherein the nutrient target is calculated based on the predicted nutrient to be consumed and an empirically determined nutrient to maintain value.

12. The method of claim 11, wherein the empirically determined nutrient to maintain value is retrieved from a non-transitory storage medium.

13. The method of any one of claims 1 to 12, wherein the nutrient is selected from glucose, glutamate, galactose, lactate, and glutamine.

14. The method of any one of claims 1 to 12, wherein the nutrient includes one or more monosaccharides.

15. The method of any one of claims 1 to 12, wherein the residual nutrient measurement comprises assaying a nutrient concentration in the bioreactor.

16. The method of any one of claims 1 to 12, wherein the residual nutrient measurement comprises performing one or more of offline nutrient measurement and inline nutrient measurement.

17. The method of any one of claims 1 to 12, wherein the residual nutrient measurement is performed by one or more of the following: a NovaFlex device and a Raman Probe.

18. The method of any one of claims 1 to 12, wherein the bioreactor is one or more of the following: a Chinese hamster ovary (CHO) cell bioreactor and a 5 L bioreactor.

19. The method of any one of claims 1 to 12, wherein cells in the bioreactor are mammalian cells.

20. The method of claim 19, wherein the cells are CHO cells.

21. A method of controlling a glucose feed in a cell culture process, comprising:
receiving a sample from a bioreactor comprising a cell culture;
measuring a residual amount of glucose from the received sample;
determining a viable cell density on a current day based on the measured residual amount of glucose;
calculating a cellular growth rate between the current culture day and a previous day based at least on the determined viable cell density on the current day;
predicting a viable cell density for a next day based at least on the calculated cellular growth rate;
predicting an integrated viable cell density for the next day based at least on the predicted viable cell density for the next day;
calculating a glucose target for the next day based at least on the predicted integrated viable cell density for the next day; and
feeding glucose to the bioreactor according to the calculated glucose target for the next day.
